(19) United States
(12) Patent Application Publication
(10) Pub. No.: US 2009/0291140 A1
(43) Pub. Date: Nov. 26, 2009

(54) TREATMENT OF DYSMENORRHEA VIA TRANSDERMAL ADMINISTRATION OF NONSTEROIDAL ANTI-INFLAMMATORY DRUGS

(76) Inventor: Andrew Korey, Los Altos Hills, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/467,649

(22) Filed: May 18, 2009

Related U.S. Application Data
(60) Provisional application No. 61/055,061, filed on May 21, 2008.

(51) Int. Cl. A61K 9/10; A61K 31/192

(52) U.S. Cl. 424/484; 514/570

(57) ABSTRACT

Methods and compositions are provided for the treatment of a subject suffering from dysmenorrhea, including both primary and second dysmenorrhea. Aspects of the invention include transdermally administering to the subject an effective amount of a nonsteroidal anti-inflammatory agent. Also provided are transdermal NSAID formulations and kits including the same that find use in practicing the subject methods.

### CROSS-REFERENCE TO RELATED APPLICATIONS

Pursuant to 35 U.S.C. §119 (e), this application claims priority to the filing date of U.S. Provisional Patent Application Ser. No. 61/055,061 filed May 21, 2008; the disclosure of which is herein incorporated by reference.

### INTRODUCTION

Pain, whether acute, chronic, or recurring, is a major source of morbidity and disability, costing billions of dollars annually in both direct and indirect costs. In women, pelvic pain is by far the most common type of pain complaint for which treatment is sought.

In making a diagnosis and treating the patient with pelvic pain, it is important to differentiate between acute and chronic pain, and the recurrent symptoms of dysmenorrhea, or painful menstruation.

The incidence of dysmenorrhea in general is difficult to estimate; however it is estimated that between ten and fifteen per cent of women suffer sufficient disability as a result of dysmenorrhea that they lose time from work, school, or home on a monthly basis. Dysmenorrhea may be classified as primary or secondary dysmenorrhea. Primary dysmenorrhea occurs because of an increase in uterine prostaglandin F2a, an increased sensitivity to prostaglandins, or both, and is more common among younger patients. Secondary dysmenorrhea is secondary to identifiable pathological or iatrogenic conditions acting on the uterus, tubes, ovaries, or pelvic peritoneum, and is more common in older patients.

A variety of therapeutic agents have been developed for use in the treatment of patients suffering from pelvic pain. Oral administration of many agents such as aspirin, acetaminophen, and NSAIDs (e.g., ibuprofen and naprosyn) can have side effects including stomach upset, gastrointestinal bleeding and ulceration, and liver and kidney damage. Drugs such as calcium antagonists (Nifedipine), or spasmolytic agents (Isoxuprine, Papaverine, Ritodrine) may suppress uterine activity, but because of their side effects, these agents have limited clinical usefulness.

### SUMMARY

Methods and compositions are provided for the treatment of a subject suffering from dysmenorrhea, including both primary and second dysmenorrhea. Aspects of the invention include transdermally administering to the subject an effective amount of a nonsteroidal anti-inflammatory agent. Also provided are transdermal NSAID formulations and kits including the same that find use in practicing the subject methods.

### DEFINITIONS

In describing and claiming the present invention, the following terminology will be used in accordance with the definitions set out below.

The terms “active agent”, “pharmacologically active agent”, or “drug” as used herein refer to a compound or composition of matter which, when administered to an organism (human or animal) induces a desired pharmacologic and/or physiologic effect by local and/or systemic action. The active agents herein are nonsteroidal anti-inflammatory drugs (NSAIDS) and pharmacologically acceptable salts, bases, esters, amides, derivatives or prodrugs thereof.

By “treatment” is meant at least an amelioration of the symptoms associated with the pathological condition afflicting the subject, where amelioration is used in a broad sense to refer to at least a reduction in the magnitude of a parameter, e.g., symptom, associated with the pathological condition being treated, such as the degree of pain, or other associated side effects. The effect may be prophylactic in terms of completely or partially preventing a disease or symptom thereof and/or may be therapeutic in terms of a partial or complete cure for a disease and/or adverse affect attributable to the disease. The present method of “treating” a patient, as the term is used herein, thus encompasses both prevention of a disorder or symptom (e.g. pelvic pain) in a predisposed individual and treatment of the disorder or symptom in a clinically symptomatic individual.

By “reduction in pain” is meant a decrease in the level or severity of pain experienced by a subject, as assessed by pain assessment tools as are known in the art and as are disclosed below. A “pain reduction of at least 50%” for example, means that the subject experiences and/or reports a level or severity of pain that is less than half of an initial level of pain experienced by the subject, as assessed by any of the suitable pain assessment methods as disclosed below.

By “therapeutically effective” or “effective amount” is meant a nontoxic but sufficient amount of an active agent (e.g. nonsteroidal anti-inflammatory agent) given to a subject to provide the desired therapeutic effect. An effective amount will be a dosage sufficient for reduction or cessation of pelvic pain. The effective amount will vary with the age and physical condition of the subject, the severity of the pain being treated, the nature of any underlying condition being treated, the duration of the treatment, the nature of any concurrent treatment, the pharmaceutically acceptable carrier used if any, and analogous factors within the knowledge and expertise of those skilled in the art.

By “transdermal” drug delivery is meant administration of a drug (i.e., active agent) to the skin surface of an individual so that the drug passes through the skin tissue and into the subject's bloodstream, thereby providing a therapeutic effect.

By “area” of skin, which refers to the area of through which active agent composition is delivered, is intended a defined area of intact unbroken living, where the skin is keratnized skin. A given area of skin may range from 1 cm² to 200 cm², such as from 2 cm² to 100 cm², and including from 4 cm² to 50 cm². The term “body surface” is used to refer to skin tissue, and specifically keratinized skin.

“Unit dose” or “unit dosage form,” as used herein, refers to physically discrete units suitable as unitary dosages for human subjects, each unit containing a predetermined quantity of drug (i.e., pharmacological agent) calculated in an amount sufficient to produce the desired effect in association with a pharmaceutically acceptable diluent, carrier or vehicle. The specifications for the unit dosage forms of pharmacological agents of the present invention depend on, for example, the particular pharmacological agent(s) employed and the effect to be achieved, the pharmacodynamics associated with the particular pharmacological agent(s) in the subject, etc.

By “pharmaceutically acceptable carrier” is meant a component such as a carrier, diluent, excipient, and the like of a composition that is compatible with the one or more pharmacological agents and other optional ingredients of the subject active agent compositions in that a pharmaceutically acceptable carrier may be combined with the pharmacological agent(s) without eliminating the biological or therapeutically effective activity of the one or more pharmacological agents, and is suitable for use in subjects as provided herein without undue adverse side effects (such as toxicity, irritation, or allergic response). Side effects are “undue” when their risk outweighs the benefit provided by the pharmaceutical agent.

### DETAILED DESCRIPTION

Methods and compositions are provided for the treatment of a subject suffering from dysmenorrhea, including both primary and second dysmenorrhea. Aspects of the invention include transdermally administering to the subject an effective amount of a nonsteroidal anti-inflammatory agent. Also provided are transdermal NSAID formulations and kits including the same that find use in practicing the subject methods.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It should be noted that, as is conventional in drawing some chemical structures, some of the hydrido groups are omitted from the drawn structures for clarity purposes, but should be understood to be present, e.g. where necessary to completely fill out the valence bonding of a carbon in a drawn structure.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Methods

As summarized above, methods of treating a subject for dysmenorrhea are provided. Subjects treated by the invention are generally mammalian subjects, more specifically female mammalian subjects, e.g., female human subjects. Dysmenorrhea treated by the methods of the invention includes both primary and second dysmenorrhea, e.g., as described in greater detail below.

Aspects of methods of invention include topically applying to a skin site of a subject a transdermal nonsteroidal anti-inflammatory drug composition (transdermal NSAID composition) in a manner sufficient to administer to said subject an effective amount of an NSAID to treat said subject for said dysmenorrhea. In methods of the invention, the transdermal NSAID composition (embodiments of which are described in greater detail below) is applied to a skin site of the subject, such as keratinized skin site of the subject.

Following application of the composition to the skin site, the topical composition is maintained at the skin site for a duration (i.e., period of time) sufficient to administer an effective amount of the NSAID to the subject. In certain embodiments, the topical formulation as maintained at the skin site for a period of time ranging from 6 hours to 7 days, such as from 12 hours to 3 days, including from 1 to 2 days.

Following application of the transdermal NSAID composition to the subject, the subject will experience a statistically significant reduction in pain, including pelvic pain, as compared to an untreated control. The assessment of pain according to the methods in the subject invention can include but is not limited to assessment tools such as the McGill Pain Questionnaire (MPQ), which can include a pain response index (PRI) and a present pain index (PPI), a visual analog scale (VAS), a numerical scale, a categorical scale, a pain faces scale, and the like. Similar pain assessment tools and surveys as known in the art may also be used. In addition to pelvic pain assessment, evaluation can also in some embodiments include the assessment the presence and severity or intensity of associated symptoms, such as backache and nausea, for example. In some embodiments, pain assessment can also include objective measures as are known in the art such as measurement of physiological parameters such as blood pressure, or by using sensors to measure a physiological parameter, etc.

In assessing the degree of pain experienced by a subject, timing of the onset of pain relief, the degree of pain reduction, and the duration of pain relief using the compositions and methods of the subject invention can be evaluated. For example, the level of pain experienced by a subject can be assessed before administration of the subject compositions, as well as after administration of the subject compositions, such as at 15 minutes, 30 minutes, 60 minutes, 120 minutes, etc.

If the target symptom(s), e.g., pelvic pain, recurs following removal of the transdermal composition, a new transdermal composition may be applied. The process may be repeated as necessary and desired to achieve treatment of the target dysmenorrhea condition, e.g., pain relief.

In some embodiments, penetration of the nonsteroidal anti-inflammatory agent employed in the subject methods is a controlled-release formulation, and the subject experiences relief from the pain for a period of hours after application of the composition. In some embodiments, the composition comprises a formulation of active agent which combines both rapidly absorbed and controlled-release formulations.

The skin site to which a composition of the invention is applied may vary, so long as application of the composition to the skin site results in sufficient administration of the NSAID active agent to the subject so that the subject is treated for the target dysmenorrhea condition. In certain embodiments, the skin site is a torso skin site, e.g., back, chest, abdomen, etc., where in certain embodiments the skin site is an abdomen skin site.

The amount of composition applied to the skin site may vary. For example, where the topical application is a patch, solution, gel, lotion, cream, foam, or aerosol applied to the abdomen, the area covered by the applied composition in the form of a patch can cover from 1 to 200 cm², such as from 2 to 100 cm², including from 4 to 50 cm² of the skin site, such as abdomen skin site, of the subject. Where desired, compositions may include a covering optionally applied thereto. Conveniently, the composition may be provided in a unit dosage format.

Application of the transdermal NSAID composition to the subject results in a therapeutic effect of the NSAID(s) sufficient to the subject to treat the subject for the target dysmenorrhea condition.

In some embodiments, a subject can apply the subject compositions at the onset of menstruation or at the onset of symptoms, in order to treat the symptoms of the target dysmenorrhea condition (e.g., pelvic pain). In other embodiments, a subject can apply the subject compositions before the onset of menstruation or before the onset of symptoms, in order to prevent the symptoms (e.g., pelvic pain) from occurring. In certain embodiments, the subject has calculated or determined when menstruation will commence, and will apply a transdermal NSAID composition to a skin site a period of time prior to onset of menstruation, e.g., 2 days or more days prior to menstruation onset, such as 1 day or more days prior to menstruation onset.

In certain embodiments, the methods of the subject invention include diagnosing a subject for the presence of the target dysmenorrhea condition. The diagnostic protocol employed may vary. In certain embodiments, the diagnostic protocol may include a complete history and physical examination, as well as tests including blood tests, urinalysis, imaging tests, and in some cases diagnostic and/or therapeutic procedures such as laparoscopy. Evaluating a subject can include determining the timing, nature, and severity of the pain both before and after treatment. Where the target system of the dysmenorrhea condition is pain, such as pelvic pain, the pelvic pain may be acute, chronic, and/or recurrent, e.g., exacerbated during menses, i.e., symptoms of primary or secondary dysmenorrhea. The methods and compositions of the subject invention can be used for treatment of pelvic pain with both primary and secondary dysmenorrhea.

Primary dysmenorrhea is more common among younger patients, whereas secondary dysmenorrhea becomes more common in older patients. Evidence suggests that primary dysmenorrhea occurs because of either an increase in uterine prostaglandin F2a, an increased sensitivity to prostaglandins, or both. Secondary dysmenorrhea is caused by, or is secondary to, identifiable pathological or iatrogenic conditions acting on the uterus, tubes, ovaries, or pelvic peritoneum. Pain results when these processes alter pressure in or around the pelvic structures, change or restrict blood flow, or cause irritation of the pelvic peritoneum. These processes may act in combination with the normal physiology of menstruation to create discomfort, or they may act independently with their symptoms becoming noticeable during menstruation. When symptoms occur between menstrual periods, these processes can result in a source of chronic pelvic pain.

Causes of secondary dysmenorrhea and chronic pain may be broadly classified as being intrauterine or extrauterine. Almost any process that can affect the pelvic viscera and cause acute pain can be a source for chronic pain or secondary dysmenorrhea. Possible intrauterine causes include but are not limited to: adenomyosis, myomas, polyps, intrauterine contraceptive devices (IUCD), infection, and benign conditions such as cervical stenosis. Possible extrauterine causes include but are not limited to: endometriosis, benign and malignant tumors, cysts, inflammation or infection from various causes, adhesions, patients who have been diagnosed with “psychogenic” pain, and pelvic congestion syndrome.

Adenomyosis is a condition characterized by a benign invasion of the endometrium into the uterine musculature, often accompanied by a diffuse overgrowth of the musculature as well. This condition is reported in between 25% to 40% of hysterectomy specimens. Grossly, the uterus will be slightly enlarged and generally symmetrical. A colicky dysmenorrhea and menorrhagia are the most frequent presenting complaints for a patient with adenomyosis. The pain seen in adenomyosis is often referred to the rectum or the sacrum. Endometriosis is thought to be coexistent in about 15% of cases. The final diagnosis of adenomyosis must be made under the microscope.

Myomas, or uterine fibroids, are the most frequent occurring human tumor and are reported to occur in 20% of women over 30 years of age, and 30% of women over 40 years of age. These tumors may vary in size from very small to over 100 pounds in weight. While these tumors can occur in any part of the uterus, cervix, or the broad ligament, those most likely to be a cause of secondary dysmenorrhea are those that cause distortion of the uterus and the uterine cavity. Pain is thought to arise from disruption of the normal uterine muscular activity or from altered intrauterine pressures. The diagnosis of fibroids will generally be made based on the physical examination findings of an enlarged and distorted uterus.

Polyps are an infrequent cause for dysmenorrhea, however pedunculated polyps within the uterine cavity can be a source of menstrual pain. When large enough to be symptomatic, these growths will generally be detectable by virtue of uterine enlargement or herniation through the cervix.

A common iatrogenic cause for secondary dysmenorrhea is the intrauterine contraceptive device (IUCD). The presence of an IUCD can cause an increase in uterine activity that may be painful, especially in women who have not had children.

Infection and its consequences can result in secondary dysmenorrhea or chronic pelvic pain. When active infection is present, it will most often present in an acute manner, and will be diagnosed as discussed above. Scarring and intraperitoneal adhesions from infection can lead to restricted motion of the pelvic viscera and pain. Pain may be experienced only during menstruation, intercourse, bowel movements, or physical activity, or it may be constant and chronic in character. Diagnosis can be made with a history of pelvic infection, especially of repeated episodes, combined with a painful pelvic examination, thickening of the adnexa, and restricted motion of pelvic viscera.

Benign diseases of the vagina and cervix such as cervical stenosis are an infrequent source of menstrual or other pelvic pain. Inspection of the cervix on speculum examination can reveal the presence of a lesion. Cervical stenosis can be assessed by the use of a probe.

Extrauterine causes of pelvic pain include endometriosis, a condition in which tissue resembling the normal uterine inner lining occurs aberrantly in various locations outside the uterus. The chief locations in which endometrial implants are found are: the ovaries; uterine ligaments; rectovaginal septum; the pelvic peritoneum, tubes, rectum, sigmoid, and bladder; and more distant locations such as the umbilicus and vagina. The endometrial implants may vary from the size of a pinhead to large pelvic masses of several centimeters. Endometriosis is most common in white women between 30 and 40 years of age. While about 8-10% of patients will present with acute symptoms, most present with severe dysmenorrhea, with pain referred to the back and rectum. The presence of nodules in the uterosacral area, in a patient that otherwise clinically presents like a patient with chronic pelvic inflammatory disease, raises the possibility of endometriosis.

Tumors that are either benign or malignant, arising in or spreading to the uterus or adnexal structures may be a cause of dysmenorrhea or pelvic pain. The presence of a mass on pelvic examination should prompt the physician to consider the possibility of a tumor.

Chronic inflammation can be a source for chronic pelvic pain and dysmenorrhea. This may occur because of the active effects of inflammation, or by virtue of the scarring from past infection. Adhesions arising from prior inflammatory processes or surgical intervention can be a source for chronic pelvic pain or dysmenorrhea. The patient's history is helpful in evaluating this possible cause.

In approximately 5-10% of patients with chronic pelvic pain, no definite cause can be identified. In some cases, these patients are diagnosed with “psychogenic” dysmenorrhea or chronic pelvic pain. In these patients, the pain itself can become the disease. Only after other physical causes have been eliminated can this diagnosis be made.

Pelvic congestion syndrome can be seen in the presence of ovarian and pelvic varicose veins, similar to varicose veins in the legs. Additionally, the abdominal wall, bladder, rectum, sigmoid, and skeletal elements of the pelvis can all be a potential source for acute or chronic pelvic pain. Each of these areas should be included in both the history and physical evaluation of the patient with the complaint of pelvic pain.

The conditions discussed above are possible causes to be considered in the diagnosis of secondary dysmenorrhea. In evaluating a subject, for example, complaints of heavy menstrual flow combined with pain suggests possible adenomyosis, myomas, or polyps. The complaint of pelvic heaviness, or change in abdominal contour, should raise the possibility of intra-abdominal neoplasia. Fever, chills, and malaise should suggest an inflammatory process. The coexisting complaint of infertility may suggest that endometriosis is a possibility, etc.

The incidence of primary dysmenorrhea is greatest in women in their late teens to early 20's. It is uncommon for true primary dysmenorrhea to occur during the first three to six menstrual cycles of a young woman. The incidence declines with age, but even women in their 40's may be affected. Childbearing does not appear to affect the incidence.

The pain of primary dysmenorrhea is often greater than that experienced with secondary dysmenorrhea. In addition to pain, these patients often experience debilitating nausea, vomiting, diarrhea, and symptomatic vasoconstriction. For the women who suffer from primary dysmenorrhea, this can be the source of significant disruption in their lives. Pain typically begins just before or after the onset of menstruation, and lasts for approximately 48 to 72 hours. The pain is often most severe on the first or second day of menstruation.

Evidence suggests that primary dysmenorrhea occurs because of either an increase in uterine prostaglandin F2a, an increased sensitivity to prostaglandins, or both. Prostaglandin F2a is a potent uterine muscle stimulator. Increased levels of prostaglandin F2a lead to an increase in uterine contractile activity, ischemia, and pain. Prostaglandin F2a is also a potent stimulator of the smooth muscle of the gastrointestinal tract, leading to the symptoms of nausea, vomiting, and diarrhea that are often experienced.

Prostaglandins are derivatives of fatty acids commonly found in the cell wall. Prostaglandin production in the uterus increases under the influence of progesterone, reaching a peak at, or soon after, the start of menstruation. Once menstruation begins, formed prostaglandins are released from the shedding endometrium. In addition, the necrosis of endometrial cells provides increased substrate for the synthetic process. Two main prostaglandins are made in the uterus: Prostaglandin F2a and Prostaglandin E2. Prostaglandin F2a is a potent smooth muscle stimulator and vasoconstrictor. Prostaglandin E2 is a potent vasodilator and platelet disaggregator. Prostaglandin E2 has been implicated as a cause of primary menorrhagia.

The increase in the force of uterine contraction in patients with primary dysmenorrhea can be striking. During normal menstruation, contractions which generate pressure of 50 to 80 mmHg, and last 15 to 30 seconds, are not uncommon. These generally occur with a frequency of between one to four contractions in ten minutes. Normal resting pressure in the uterus is generally five to 15 mmHg. In women with dysmenorrhea, however, contractions may reach peak pressures in excess of 400 mmHg, and last longer than 90 seconds. Contractions can also be more frequent, with less than 15 seconds between contractions. Baseline pressure in the uterus can sometimes be as high as 80 to 100 mmHg. Pressures of this magnitude and duration can cause significant ischemia. The exact mechanism that creates the sensation of pain is unknown. Recent studies show a strong correlation between pain and pain relief, and the parameters of uterine work, maximal pressures, frequency and quality of contractions, rate of pressure change, and the quality of “rest” between uterine contractions.

Patients with primary dysmenorrhea generally present with the complaint of recurrent, month after-month, spasmodic lower abdominal pain occurring on the first one to three days of menstruation. The pain is diffusely located in the suprapubic area with radiation around and through to the back. The labor-like pain is described as “coming and going,” and the patient will often use a first opening and closing to illustrate their description. This pain is often accompanied by moderate to severe nausea. Vomiting and/or diarrhea are not infrequent. Patients often double up into a fetal position in an effort to gain relief. Many patients will report having tried a heating pad or hot water bottle in an effort to decrease their discomfort.

The physical examination will generally provide clues to the diagnosis, if not the diagnosis itself, in most patients with the complaint of dysmenorrhea or chronic pelvic pain. The presence of asymmetrical, or irregular enlargement of the uterus should suggest myomas, or other tumors. Symmetrical enlargement of the uterus is often present in cases of adenomyosis, and occasionally when intrauterine polyps are present. The presence of painful nodules in the posterior cul-de-sac and restricted motion of the uterus are suggestive of endometriosis. Restricted motion of the uterus is also found in cases of pelvic scarring from adhesions, or inflammation. Inflammatory processes often cause thickening of the adnexal structures. This thickening may be palpable on physical examination. The physical examination of a patient with primary dysmenorrhea should be normal. There should be no palpable abnormalities of the uterus or adnexa. Speculum and abdominal examinations should similarly be normal. If the patient is examined during the time of actual symptoms they are often found to be pale and “shocky.” The diagnosis of primary dysmenorrhea however should not be made without thoroughly evaluating and eliminating other possible causes.

The laboratory evaluation of the patient with secondary dysmenorrhea or chronic pelvic pain can include blood tests such as hematocrit to evaluate for excessive blood loss. Sedimentation rates can help to identify a chronic inflammatory processes. Radiological evaluation of the patient with xrays, CT, MRI, etc. can help detect the presence of both gynecological and non-gynecological sources of pain, such as from the gastrointestinal or urinary tract. Ultrasound examinations of the pelvis can demonstrate the presence and extent of myomas, adnexal and other tumors, or locate an intrauterine IUCD, for example. In many cases of pelvic pain, laparoscopic examination of the pelvic organs is needed for additional diagnostic information and/or therapy. In the patient with primary dysmenorrhea, laboratory and/or imaging tests are usually normal, and are primarily of value in excluding causes of secondary dysmenorrhea.

Once primary or secondary dysmenorrhea has been diagnosed and any treatable causes of pain have been evaluated and treated, the pain associated with dysmenorrhea can be treated using the nonsteroidal anti-inflammatory compositions and methods of the subject invention, e.g., as described above.

Transdermal compositions employed in the subject methods will include a nonsteroidal anti-inflammatory drug as the active agent present in a transdermal composition. In some embodiments, two or more different nonsteroidal anti-inflammatory agents may be present in the subject compositions. In some embodiments, the non-steroidal anti-inflammatory agent (e.g., flurbiprofen) is the only active agent present in a transdermal composition. For example, the transdermal composition can include a non-steroidal anti-inflammatory agent which, when administered in a topical formulation, can penetrate the skin surface such that an effective amount of the nonsteroidal anti-inflammatory agent reaches the bloodstream without needing an additional agent, such as a permeation enhancing agent (i.e., an agent for providing increased skin permeability). Therefore, in some embodiments the transdermal composition can include a non-steroidal anti-inflammatory agent without a permeation enhancer (e.g., a hydrophilic agent such as a hydroxide-releasing agent, or a lipophilic enhancer or co-enhancer, such as such as a fatty alcohol, a fatty ether, or a fatty acid ester, including fatty acid esters of polyols such as propylene glycol and glycerol, or a permeation enhancer comprised of both a hydrophilic component and a lipophilic component).

In some embodiments, the transdermal composition can include a non-steroidal anti-inflammatory agent (e.g., flurbiprofen) as the only active agent present in an adhesive composition (e.g., an adhesive mass, as in Example I). The non-steroidal anti-inflammatory agent (e.g., flurbiprofen) in an adhesive composition can further be spread on a film, such as a polyethyleneterephthalate film, and can further have a backing layer (e.g., a polyester woven fabric or non-woven fabric). In some embodiments, the transdermal composition can consist essentially of a non-steroidal anti-inflammatory agent (e.g., flurbiprofen) in an adhesive composition which is spread on a film which is further placed on a backing. The nonsteroidal anti-inflammatory agent employed in the subject methods will be a nonsteroidal anti-inflammatory agent which, when administered in a topical formulation, can penetrate the skin surface such that an effective amount of nonsteroidal anti-inflammatory agent reaches the bloodstream, resulting in the reduction of pelvic pain in the subject.

Any suitable nonsteroidal anti-inflammatory compositions can be used in the methods and compositions of the subject invention, such as those disclosed in the exemplary families of nonsteroidal anti-inflammatory drugs as shown in Table 1, below.

TABLE 1
Exemplary Families of Nonsteroidal Anti-inflammatory Drugs
DRUG

CARBOXYLATES
Salicylic Acids:
Acetylsalicylic acid
Diflunisal
Salicylate
Indoleacetic Acids:
Diclofenac Potassium
Diclofenac Sodium
Etodolac
Indomethacin
Ketorolac Tromethamine
Sulindac
Tolmetin
Propionic Acids:
Fenoprofen Calcium
Flurbiprofen
Ibuprofen*
Ketoprofen
Naproxen sodium*
Naproxen*
Fenamates:
Meclofenamate sodium*
Mefenamic acid*
ENOLIC ACIDS
Pyrazolones
Oxyphenbutazone
Phenylbutazone
Nabumetone
Celecoxib
Refecoxib*
Oxicams
Piroxicam
Meloxicam
*FDA approved for primary dysmenorrhea Modified from: Smith RP: Gynecology in Primary Care. Williams and Wilkins, Baltimore, Maryland, 1996, p. 399.

There are two broad classes of NSAID compounds, each with sub-groups as shown in Table 1. Drugs of the enolic acid type appear to be primarily Type II inhibitors of prostaglandin synthesis. These agents act through the inhibition of the isomerase/reductase step in the formation of PGE2 and PGF2a. The most frequently used agents in the enolic acid groups are phenylbutazone and piroxicam. There are differences among these agents with respect to half-life, side effects, etc.

The carboxylates are the most commonly used agents for pain relief including dysmenorrhea. Within this major group there are four families of compounds that have individual characteristics. The salicylic acids and esters appear to inhibit cyclo-oxygenase by the donation of their acetyl group to, the enzyme. Increased potency is seen in the acetic acid groups. While sulindac (Clinoril) must undergo reduction to a sulfide form before becoming active, most of the drugs in this group are effective as anti-inflammatory and analgesic agents. In several studies, indomethacin has shown usefulness in treating dysmenorrhea, but a moderate incidence of side effects with oral administration has limited the use of this and most other drugs in this class for treating dysmenorrhea.

The most commonly used drugs for dysmenorrhea come from two classes: arylalkanoic acids (propionic acid derivatives) and anthranilic acids (fenamates). Both ibuprofen (Motrin, Rufen) and naproxen (Naprosyn, Anaprox) are commonly used for dysmenorrhea. Other drugs of this class (benoxaprofen, ketoprofen, fenoprofen) have been used for pain relief or arthritis therapy. Ibuprofen was the first drug of this class to be studied in dysmenorrhea and has shown effectiveness in subsequent subjective studies. The subjective studies of naproxen and naproxen sodium have shown good pain relief in dysmenorrhea, even in the presence of intrauterine devices. In this country, mefenamic acid (Ponstel) is approved for dysmenorrhea and clinical studies supporting the use of meclofenamate (Meclomen) are well under way. New in-vitro studies have shown meclofenamate to inhibit the activity of 5-lipoxygenase.

Any of the nonsteroidal anti-inflammatory agents disclosed above or in the table in Table 1 can be used in the methods and compositions of the subject invention. The nonsteroidal anti-inflammatory agent of the composition can be a carboxylate or enolic acid compound. Carboxylate compounds can include but are not limited to salicylic acids, indoleacetic acids, propionic acids, and fenamates. Enolic acid compounds can include but are not limited to pyrazolones and oxicams. Compounds that can be used in the present invention include, but are not limited to: propionic acid derivatives such as ketoprofen, flurbiprofen, ibuprofen, naproxen, fenoprofen, benoxaprofen, indoprofen, pirprofen, carprofen, oxaprozin, pranoprofen, suprofen, alminoprofen, butibufen, fenbufen and tiaprofenic acid; acetylsalicylic acid; apazone; diclofenac; difenpiramide; diflunisal; etodolac; flufenamic acid; indomethacin; ketorolac; meclofenamate; mefenamic acid; nabumetone; phenylbutazone; piroxicam; salicylic acid; sulindac; tolmetin; and combinations of any of the foregoing.

In some embodiments, the composition may comprise more than one nonsteroidal anti-inflammatory agent. In some embodiments, pharmaceutically acceptable analogs of such NSAIDs can be used as well, including salts, esters, amides, prodrugs or other derivatives. In certain embodiments, the nonsteroidal anti-inflammatory agent is present in the composition as a free base to promote penetration of the agent through the skin surface.

The amount of nonsteroidal anti-inflammatory agent present in the subject compositions will be sufficient to provide an effective amount of the agent when topically administered according to the subject methods. The precise amount of nonsteroidal anti-inflammatory agent present in the transdermal formulation will depend on the particular agent employed, and may range from 0.1 to 50% (w/w), such as from 0.5 to 20% (w/w), including 1 to 10% (w/w), including 1 to 5% (w/w).

In some embodiments, the formulation of the subject compositions is a rapid absorption formulation, such that the absorption rate of the active agent i.e., the nonsteroidal anti-inflammatory active agent, is rapidly absorbed across the skin surface into a subject's systemic circulation. In some embodiments, the formulation of the subject compositions is a controlled-release formulation, such that the absorption rate of the active agent i.e., the nonsteroidal anti-inflammatory active agent is released at a specific rate or rates over time.

In some embodiments, the formulation of the subject compositions is a combination of a rapid-absorption and a controlled-release formulation, such that the nonsteroidal anti-inflammatory active agent is rapidly absorbed across the skin surface into a subject's systemic circulation, and the active agent is also released at a specific rate or rates over time.

The topical nonsteroidal anti-inflammatory agent composition can be in the form of a patch, cream, lotion, foam, ointment, paste, solution, gel, emulsion, suspension, solution, applicator stick, jelly, paint, powder, aerosol spray, or may be prepared with liposomes, micelles, or microspheres. In some embodiments, the method may involve use of a drug delivery device, or methods for physically enhancing skin permeation such as, for example, electrophoretic techniques such as iontophoresis, or phonophoresis (the use of ultrasound) to increase physical penetration of the active agent composition. In some embodiments, when administered in a topical formulation, the transdermal composition can penetrate the skin surface such that an effective amount of the nonsteroidal anti-inflammatory agent reaches the bloodstream without needing a drug delivery device or method for physically enhancing skin permeation. In certain embodiments, one or more pharmacological agents may be administered via a transdermal patch or film system such as or analogous to that described, e.g., in U.S. Pat. Nos.: 6,645,520, 6,503,532; 5,302,395; 5,262,165; 5,248,501; 5,232,702; 5,230,896; 5,227,169; 5,212,199; 5,202,125; 5,173,302; 5,154,922; 5,139,786; 5,122,383; 5,023,252; 4,978,532; 5,324,521; 5,306,503; 5,302,395; 5,296,230; 5,286,491; 5,252,334; 5,248,501; 5,230,896; 5,227,169; 5,212,199; 5,202,125; 5,173,302; 5,171,576; 5,139,786; 5,133,972; 5,122,383; 5,120,546; 5,118,509; 5,077,054; 5,066,494; 5,049,387; 5,028,435; 5,023,252; 5,000,956; 4,911,916; 4,898,734; 4,883,669; 4,882,377; 4,840,796; 4,818,540; 4,814,173; 4,806,341; 4,789,547; 4,786,277; 4,702,732; 4,690,683; 4,627,429; and 4,585,452, the disclosures of which are herein incorporated by reference.

Embodiments of interest include a pharmacological agent formulation in the form of a discrete patch or film or plaster or “adhesive mass”, or the like adapted to remain in intimate contact with the epidermis of the recipient for a period of time. For example, such transdermal patches may include an adhesive matrix layer, e.g., polymeric layer, or “reservoir layer”, in which one or more pharmacological agent(s) are retained. The adhesive matrix layer, when present, comprises adhesives suitable for use medical applications, such as polymeric adhesives, including but not limited to, e.g., acryl-type, synthetic rubber-type, and natural rubber-type materials. In some embodiments, the pharmacological agent formulation in the form of a plaster, or adhesive mass, can be spread on a film, such as a polyethyleneterephthalate (PET) film. In this embodiment, the adhesive composition containing the non-steroidal anti-inflammatory agent can have a thickness of from 30 to 400 μm, such as from 50 to 300 μm, or 70 to 250 μm.

In some embodiments, the adhesive is a copolymer of alkyl(meth)acrylates, present in an amount of 40 wt % or more. In some embodiments, a copolymer of one type or two types or more of alkyl(meth)acrylates and one type or two types of more of copolymerized monomer is used, in some embodiments, a copolymer of one type or two types or more of alkyl(meth)acrylates is present in an amount of from about 50 wt % to about 98 wt %; and one type or two types of more of copolymerized monomer is present in an amount of from about 2 wt % to about 50 wt %. Suitable alkyl(meth)acrylates include esters of from a primary to a tertiary alcohol, e.g., where the carbon number of the aikyl group is from 2 to 18, or from 4 to 12. In some embodiments, acrylic acid or methacrylic acid is used. Suitable copolymerized monomers generally have at least one unsaturated double bond that participates in the copolymerization reaction, or a monomer that has functional groups on the side chain. Functional groups include, e.g., a carboxyl group such as (meth)acrylic acid, itaconic acid, maleic acid, suifoxyl group such as styrene sulfonic acid, sulfopropyl(meth)acrylate, allylsulfonic acid; a hydroxyl group such as (meth)hydroxyethyl acrylate, (meth)hrdroxypropyl acrylate; an amino group such as aminoethyl(meth)acrylate, dimethylaminoethyl(meth)acrylate; an amide group such as (meth)acrylamide, dimethyl(meth)acrylamide, N-butyl acrylamide; and an alkoxyl group such as methoxyethyl(meth)acrylate, methoxyethylene glycol(meth)acrylate, methoxy polyethylene glycol(meth)acrylate. Other monomers that are suitable for copolymerization include, but are not limited to, N-vinyl-2-pyrrolidone, methyl vinyl pyrrolidone, (meth)acrylonitrile, vinyl acetate, vinyl propionate, vinyl piridine, vinyl piperidone, vinyl pyrimidine, vinyl piperadine, vinyl pyrazine, vinyl pyrrol, vinyl imidazole, vinyl caproiactam, vinyl oxazole, and vinyl morpholine. Suitable acryl-type adhesives include, but are not limited to, acrylic acid-octylacrylate copolymer; 2-ethylhexyl acrylate-vinyl pyrrolidone copolymer solution; 2-methoxyethyl acrylate-vinyl acetate copolymer; 2-ethylhexyl acrylate-2-ethylhexyl methacrylate-dodecyl methacryiate copolymer; and methyl acrylate-2-ethylhexyl acrylate copolymer resin emulsion.

Also of interest are synthetic rubber adhesives. Suitable synthetic rubber-type adhesives include, but are not limited to, styrene-isoprene-styrene block copolymer, polyisobutylene, isoprene rubber, styrene-butadiene-styrene block copolymer, styrene-butadiene rubber, and silicon rubber. The adhesive will in some embodiments comprise one type of synthetic rubber. In other embodiments, the adhesive will include two or more types of synthetic rubber. In some embodiments, a synthetic rubber-type adhesive or a natural rubber-type adhesives will have low adhesion. In these embodiments, one or more adhesion enhancers will be added to enhance adhesion. Suitable adhesion enhancers include, but are not limited to, polyterpene resin type, petroleum resin type, rosin type, rosin ester type, and oil-soluble phenol.

The matrix layer may be operatively associated with a support or backing, such as a polyester woven fabric, or a non-woven fabric. A patch may also comprise a separate non-drug containing adhesive layer, and/or a protective coating. For patch or analogous formulations, the formulations may have any convenient shape. In certain embodiments, the formulations may have a circular or oval shape. In some embodiments, the patch can be cut into a desired shape. In certain embodiments, the formulations may have a dark colored or black backing material. In some embodiments the active agent may be in the form of a gel, such as those disclosed in U.S. Pat. Nos. 6,346,271 and 5,897,271, incorporated herein by reference. Pharmacological agent formulations suitable for transdermal administration may also be delivered by iontophoresis and may take the form of an optionally buffered aqueous solution of the pharmacological agent compound. Suitable formulations may include citrate or bis/tris buffer (pH 6) or ethanol/water and contain a suitable amount of active ingredient.

The nonsteroidal anti-inflammatory agents of the subject compositions may be co-administered with one or more additional active agents, e.g., other pharmacologically active agents. The subject compositions may therefore optionally contain, in addition to a nonsteroidal anti-inflammatory agent, at least one other therapeutic agent useful in the treatment of a condition, e.g., pelvic pain, dysmenorrhea. Accordingly, an agent may be administered alone or with or in appropriate association, as well as in combination, with other pharmaceutically active compounds. As used herein, “administered with” means that at least one pharmacological agent and at least one other adjuvant (including one or more other pharmacological agents) are administered at times sufficiently close that the results observed are indistinguishable from those achieved when one pharmacological agent and at least one other adjuvant (including one or more other pharmacological agents) are administered at the same point in time. The pharmacological agent and at least one other adjuvant may be administered simultaneously (i.e., concurrently) or sequentially. Simultaneous administration may be carried out by mixing the at least one pharmacological agent and at least one other adjuvant prior to administration, or by administering the pharmacological agent and at least one other adjuvant at the same point in time. Such administration may be at different anatomic sites. The phrases “concurrent administration,” “administration in combination,” “simultaneous administration” or “administered simultaneously” may also be used interchangeably and mean that the at least one pharmacological agent and at least one other adjuvant are administered at the same point in time or immediately following one another. In the latter case, the at least one pharmacological agent and at least one other adjuvant are administered at times sufficiently close that the results produced are synergistic and/or are indistinguishable from those achieved when the at least one pharmacological agent and at least one other adjuvant are administered at the same point in time. Alternatively, a pharmacological agent may be administered separately from the administration of an adjuvant, which may result in a synergistic effect or a separate effect. The methods and excipients described herein are merely exemplary and are in no way limiting.

The subject composition may also comprise a pharmaceutically acceptable carrier or any other necessary components of topical, transdermal, or transmucosal formulations and delivery devices, such as solubilizing agents, suspending agents, dispersing agents, preservatives, animal and vegetable fats, oils, or waxes, stabilizing agents, thickening or gelling agents, buffering agents, or adhesive agents. Non-limiting examples of pharmaceutically acceptable components include, but are not limited to, any of the standard pharmaceutical carriers such as phosphate buffered saline solutions, water, emulsions such as oil/water emulsions or water/oil emulsions, microemulsions, and various types of wetting agents. Suitable nontoxic pharmaceutically acceptable carriers for use in the compositions of the present invention will be apparent to those skilled in the art of pharmaceutical formulations and examples are described in REMINGTON'S PHARMACEUTICAL SCIENCES, 19.sup.th Edition, A. R. Gennaro, ed., 1995. The choice of suitable carriers will depend on the exact nature of the particular dosage form desired, e.g., whether the active ingredient(s) is/are to be formulated into a cream, lotion, foam, ointment, paste, solution, or gel, as well as on the active ingredient(s).

### Utility

Methods of the invention find use in the treatment of dysmenorrhea, including primary and secondary dysmenorrhea. As reviewed above, by treatment is meant the amelioration of at least one symptom of the target dysmenorrhea condition, such as pain, e.g., pelvic pain.

In some embodiments, the methods can be used to treat a subject who has a history of moderate dysmenorrhea for at least 5 years, such as at least 8 years, or at least 10 years, etc. In other embodiments, the methods can be used to treat a subject who has a history of severe dysmenorrhea for at least 5 years, such as at least 8 years, or at least 10 years, etc. In some aspects of the invention, the methods can include treating a subject with a history of moderate or severe dysmenorrhea that has been resistant to treatment with other methods. By resistant to treatment with other methods is meant a subject with primary or secondary dysmenorrhea who has not experienced a significant amelioration of the symptoms associated with dysmenorrhea, such as the degree of pelvic pain, or cramping, etc., with other methods of treatment. As discussed above, the moderate or severe dysmenorrhea can be primary dysmenorrhea, or it can be secondary dysmenorrhea.

The methods can include a step of determining when menstruation will commence for a subject. Methods of determining when the menstrual period will start can include calculation of the starting date based on the calendar and the known length of the menstrual period for a particular subject, and can also include assessment of the onset of associated symptoms, such as headache, feeling of being bloated, nausea, breast discomfort, etc. which can be caused by premenstrual water retention or hormone fluctuation.

The methods can include treating a subject prior to the onset of the menstrual period. For example, the methods can include applying a transdermal flurbiprofen composition (e.g., flurbiprofen transdermal tape) to a skin site for a period of time prior to onset of menstruation, e.g., 1 day prior to the onset of the menstrual period, or 2 days prior to the onset of the menstrual period, e.g. The methods can also include treating a subject during the menstrual period. For example, the methods can include applying a transdermal flurbiprofen composition (e.g., flurbiprofen transdermal tape) to a skin site for a period of time during the menstrual period, such as for 2 or more days, such as 3 or more days, or 4 or more days, or 5 or more days, etc. In some embodiments, the methods can include applying a transdermal flurbiprofen composition to a skin site for a period of time prior to the onset of the menstrual period and continuing treatment for a period of time during the menstrual period.

In some embodiments, methods can include applying to a skin site of a subject suffering from moderate or severe dysmenorrhea a 3% flurbiprofen transdermal composition in a manner sufficient to treat the subject for dysmenorrhea. For example, the methods can include applying to a skin site of a subject suffering from moderate or severe dysmenorrhea a 3% flurbiprofen transdermal composition, such that the subject receives a total dose of 63 mg of flurbiprofen a day. In some embodiments, the flurbiprofen transdermal composition is flurbiprofen transdermal tape.

The methods can further be used to treat a subject with a history of moderate or severe dysmenorrhea with an effective amount of a transdermal composition, such as a transdermal flurbiprofen composition, such that pain intensity of dysmenorrhea symptoms is reduced. For example, the methods can include treating a subject with a history of moderate or severe dysmenorrhea with an effective amount of a transdermal composition, such as a transdermal flurbiprofen composition, such that pain intensity of pelvic pain is reduced. In some embodiments, the subject can experience a significant reduction in pain, such as pelvic pain, compared to the degree of pelvic pain the subject experienced during menstrual periods. prior to treatment with the-subject methods. For example, in some embodiments, applying to a skin site a transdermal flurbiprofen composition in a manner sufficient to topically administer an effective amount of flurbiprofen to treat the subject for dysmenorrhea can provide a significant reduction in pain, such as a “good” or an “excellent” reduction in the level or severity of pelvic pain experienced by the subject. In other embodiments, applying to a skin site a transdermal flurbiprofen composition in a manner sufficient to topically administer an effective amount of flurbiprofen to treat the subject for dysmenorrhea can provide a significant reduction in pain, such as a “good” or an “excellent” reduction in the severity of the most severe pelvic pain experienced by the subject.

As such, the subject compositions find use in the treatment of dysmenorrhea, either primary or secondary. The invention accordingly provides a novel and highly effective means for treatment of dysmenorrhea, including primary and secondary dysmenorrhea.

### Kits

Also provided are kits that find use in practicing the subject methods, where the subject kits at least include a transdermal nonsteroidal anti-inflammatory agent composition, as described above. The subject active agent composition in the kits may be present in a package, as described above. Kits may include the transdermal nonsteroidal anti-inflammatory agent composition in an amount suitable for a single application (e.g., a unit dose, or single dose) or multiple applications. In instances in which composition is present in a kit in an amount sufficient for more than one application, multiple packages, as described above, may be provided with each containing an amount of the transdermal nonsteroidal anti-inflammatory agent composition for a single application.

The subject kits may also include instructions for how to use the compositions in methods of delivering a nonsteroidal anti-inflammatory agent to a subject. The instructions may include information about dosing schedules etc., and/or how to use the packaged compositions. In certain embodiments, the subject kits can include instructions on how to use the compositions to treat a particular disease condition, e.g., primary dysmenorrhea. The instructions may be recorded on a suitable recording medium. For example, the instructions may be printed on a substrate, such as paper or plastic, etc. As such, the instructions may be present in the kits as a package insert, in the labeling of the container of the kit or components thereof (i.e. associated with the packaging or subpackaging) etc. In other embodiments, the instructions are present as an electronic storage data file present on a suitable computer readable storage medium, e.g. CD-ROM, diskette, etc.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### EXPERIMENTAL

### I. Flurbiprofen Tape

### A. Formulation

| | | |
|---|---|---|
| | Formula | |
| Ingredient | (mg/patch) | Formula (% w/w) |

Active Ingredient

| | | |
|---|---|---|
| Flurbiprofen | 31.5 | 3.0 |

Excipients

| | | |
|---|---|---|
| Styrene/Isoprene/Styrene | 168.00 | 16.0 |
| Block Copolymer | | |
| Hydrogenated Rosin | 441.00 | 42.0 |
| Glycerol Ester | | |
| Polybutene | 52.50 | 5.00 |
| Dibutylhydroxytoluene | 21.00 | 2.00 |
| Liquid Paraffin | 336.00 | 32.0 |
| TOTAL | 1050.00 | 100.00 |
| Woven fabric | | |
| Polyethyleneterephthalate | | |
| (PET) film | | |

### B. Fabrication

In fabricating a topical plaster composition according to the above formulation, Styrene/Isoprene/Styrene Block Copolymer, Hydrogenated Rosin Glycerol Ester, Polybutene, Dibutylhydroxytoluene and Liquid Paraffin are melted under heating. Then flurbiprofen is added to the above adhesive, and mixed under stirring, to prepare an adhesive mass for the plaster. The adhesive mass thus prepared is spread on the polyethylene terephthalate film, to form an adhesive layer having a thickness of 50 to 300 μm. The obtained adhesive layer is laminated with a polyester woven fabric or nonwoven fabric which is a backing, and then the resultant article is cut into a desired size and shape to produce the desired flurbiprofen plaster composition.

### II. Flurbiprofen Plaster in the Treatment of Dysmenorrhea

### A. Protocol

The utility and efficacy of the Flurbiprofen plaster formulation prepared as described in I above in treating or preventing menstrual pain was demonstrated in six women with moderate to severe dysmenorrhea. Six women, aged 24 to 36 years old, with a history of moderate to severe dysmenorrhea of 8 to 25 years duration, were treated with FTD (3% flurbiprofen, 63 mg daily) for 1-2 days prior to and 2-5 days after the onset of their menstrual period.

### B. Results

The results are provided in Table 2, below.

TABLE 2

| | | | Severity | | |
|---|---|---|---|---|---|
| | | Years | Previous | Subject Global | Worst |
| Subject # | Age | Dysmen | Dysmen | Assessment | pain |
| 051003 | 39 | 26 | Moderate | GOOD | Severe |
| 051004 | 31 | 20 | Moderate | GOOD | Moderate |
| 051006 | 28 | 9 | Severe | GOOD | Moderate |
| 051007 | 35 | 23 | Severe | GOOD | Moderate |
| 051008 | 36 | 24 | Severe | EXCELLENT | Mild |
| 051010 | 24 | 10 | Moderate | GOOD | Moderate |

As shown in the above table, one woman reported “Excellent” results, and five women reported “Good” results. Three of these women had a longstanding history of severe dysmenorrhea, but reported only Mild or Moderate pain while receiving treatment with the Flurbiprofen plaster formulation.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. A method for treating a subject suffering from dysmenorrhea, said method comprising:
applying to a skin site of said subject a transdermal nonsteroidal anti-inflammatory drug composition in a manner sufficient to topically administer to said subject an effective amount of a nonsteroidal anti-inflammatory drug (NSAID) to treat said subject for dysmenorrhea.

2. The method according to claim 1, wherein said subject is a female human.

3. The method according to claim 2, wherein said method further comprises diagnosing said female human as suffering from dysmenorrhea.

4. The method according to claim 2, wherein said treatment comprises reducing pain intensity of said dysmenorrhea.

5. The method according to claim 1, wherein said NSAID is present in said composition in amount ranging from 0.1 to 50% (w/w).

6. The method according to claim 1, wherein said NSAID is flurbiprofen.

7. The method according to claim 1, wherein said transdermal composition is a patch.

8. The method according to claim 7, wherein said patch comprises an adhesive matrix that comprises said NSAID.

9. The method according to claim 8, wherein said adhesive matrix comprises a synthetic rubber.

10. The method according to claim 9, wherein said synthetic rubber is SIS.

11. The method according to claim 1, wherein said skin site is an abdomen skin site.

12. The method according to claim 1, wherein said dysmenorrhea is primary dysmenorrhea.

13. The method according to claim 1, wherein said dysmenorrhea is secondary dysmenorrhea.
